(19) United States
(12) Patent Application Publication
(10) Pub. No.: US 2025/0345557 A1
(43) Pub. Date: Nov. 13, 2025

(54) DRUG-ELUTING EXPANDABLE URETHRAL BALLOON CATHETER

(71) Applicants: INJE UNIVERSITY INDUSTRY-ACADEMIC COOPERATION FOUNDATION, Gimhae-Si (KR); U-MED CO., LTD., Gimhaesi (KR)

(72) Inventor: Myungchan PARK, Busan (KR)

(21) Appl. No.: 18/868,898

(22) PCT Filed: Jun. 13, 2022
(86) PCT No.: PCT/KR2022/008328

§ 371 Date: Nov. 25, 2024

(30) Foreign Application Priority Data
May 26, 2022 (KR) 10-2022-0064705

(51) Int. Cl. A61M 25/00; A61M 25/10; A61M 39/10

(52) U.S. Cl. CPC A61M 25/0017; A61M 25/1011; A61M 39/10; A61M 2025/0001; A61M 2025/0093; A61M 2025/105; A61M 2039/1077; A61M 2210/1085; A61M 2210/1089

(57) ABSTRACT

Proposed is a drug-eluting expandable urethral balloon catheter. The drug-eluting expandable urethral balloon catheter includes a tube part inserted into the urethra, a separation prevention part including a first balloon that expands in the bladder according to the inflow of fluid to prevent separation the of tube part and maintain airtightness of the bladder, a drug injection part having a plurality of pores formed so that a drug is injected into the stricture site of the urethra, a urethra expansion part including a second balloon which is positioned on a front end of the drug injection part and inflates or contracts according to the inflow and outflow of fluid, and a branched connection part branched from one side of the tube part to be connected thereto so as to enable fluid to flow in and out of the tube part.

### TECHNICAL FIELD

The present disclosure relates to a drug-eluting expandable urethral balloon catheter.

### BACKGROUND ART

Urethral stricture is a symptom of discomfort caused by the urethra becoming narrow due to scarring that occurs during the healing process of wounds in the urethral mucosa caused by inflammation or injury. Stricture occurs inside the urethra of a patient's penis, narrowing the inside of the urethra. In the case of a patient with urethral stricture, surgery to widen the narrowed urethra is mainly performed by using an endoscopic technique.

However, once urethral stricture occurs, even if the urethra is expanded through surgery, a recurrence rate thereof is high and it is difficult to restore the urethra to its original function before damage. In general, even after treatment for urethral stricture, the recurrence rate due to fibrotic reaction is known to be 50-70%.

Therefore, in order to prevent the recurrence of stricture after surgery, a method that can easily administer various stricture prevention drugs and safely expand the urethra is required.

### DISCLOSURE

### Technical Problem

The present disclosure has been made to solve the above problems and provides a drug-eluting expandable urethral balloon catheter that enables a patient to safely perform urethral dilation and drug administration simultaneously while the catheter is in place in the urethra.

Problems that the present disclosure seeks to solve are not limited to the problems mentioned above, and other problems not mentioned will be clearly understood by those skilled in the art from the description below.

### Technical Solution

In order to accomplish the above objectives, a drug-eluting expandable urethral balloon catheter according to an embodiment of the present disclosure includes: a tube part inserted into the urethra; a separation prevention part including a first balloon that inflates in the bladder to prevent separation of the tube part and maintain airtightness of the bladder; a drug injection part having a plurality of pores formed so that a drug is injected into a stricture site of the urethra; a urethra expansion part including a second balloon positioned and inflated at a front end of the drug injection part to prevent leakage of the drug; and a branched connection part branched from one side of the tube part to be connected thereto.

In addition, the tube part may include: a urinary tube; a first fluid passage tube communicating with the first balloon; a drug injection tube communicating with the plurality of pores; and a second fluid passage tube communicating with the second balloon.

In addition, the tube part may have a hollow portion constituting the urinary tube, and each of the first fluid passage tube, the drug injection tube, and the second fluid passage tube may extend within a cross-section of the tube part.

In addition, the branched connection part may include: a first adapter connected to a urinary tube of the tube part to perform urinary catheterization from the bladder; a second adapter connected to introduce or discharge fluid, such as air or saline solution, into or out of the first balloon; a third adapter connected to inject a drug into the stricture site of the urethra through the plurality of pores; and a fourth adapter connected to introduce or discharge fluid, such as air or saline solution, into or out of the second balloon.

In addition, the branched connection part may inflate the second balloon by introducing fluid thereto through the fourth adapter when injecting a drug into the stricture site of the urethra through the third adapter.

In addition, when the second balloon of the urethra expansion part is inflated, a portion of the second balloon adjacent to the tube part may be formed to be inflated steeper than a portion of the second balloon adjacent to the drug injection part.

In addition, when the second balloon of the urethra expansion part is inflated, a thickness of a portion of the tube part connected to the second balloon may be small.

In addition, when the second balloon of the urethra expansion part is inflated, a groove may be formed in a portion of the second balloon so that the second balloon is divided into two parts.

In addition, the catheter may further include: a sensing part which senses pressure of fluid flowing into or out of the first fluid passage tube and the second fluid passage tube.

In order to accomplish the above objectives, a drug-eluting expandable urethral balloon catheter according to another embodiment of the present disclosure includes: a tube part inserted into the urethra; a separation prevention part including a third balloon that inflates in the bladder to prevent separation of the tube part and maintain airtightness of the bladder; a urethra expansion part including a fourth balloon positioned and inflated at a stricture site of the urethra to prevent leakage of a drug; and a branched connection part branched from one side of the tube part to be connected thereto, wherein the urethra expansion part has a plurality of microneedles formed on a surface of the fourth balloon.

In addition, the microneedles of the urethra expansion part may include an antifibrotic drug.

Other specific details of the present disclosure are included in the detailed description and drawings.

### Advantageous Effects

According to the present disclosure, it is possible for a patient to periodically dilate the urethra on his or her own and simultaneously administer drug.

Accordingly, it is possible to effectively prevent restenosis of the urethra after a procedure or surgical treatment for urethral stricture, such as urethral dilation or a urethrotomy.

### DESCRIPTION OF DRAWINGS

FIG. 1 is a drawing illustrating a drug-eluting expandable urethral balloon catheter according to an embodiment of the present disclosure.

FIG. 2 is a drawing illustrating a longitudinal cross-section of the urethral catheter of FIG. 1.

FIG. 3 is a drawing illustrating the A-B cross-section of a tube part of the urethral catheter of FIG. 2.

FIG. 4 is a drawing illustrating the body into which the urethral catheter is inserted, and FIG. 5 is a drawing illustrating a state in which the urethral catheter is inserted.

FIG. 6 is a drawing illustrating a sensing part installed in the urethral catheter of FIG. 1.

FIG. 7 is a drawing illustrating the detailed configuration of the sensing part of FIG. 6.

FIGS. 8a to 8c are drawings illustrating several embodiments of a second balloon of the urethral catheter of FIG. 1.

FIG. 9 is a drawing illustrating a drug-eluting expandable urethral balloon catheter according to another embodiment of the present disclosure.

FIG. 10 is a drawing illustrating a cross-section of the tube part of the urethral catheter of FIG. 9.

FIG. 11 is a drawing illustrating microneedles formed in a urethra expansion part of the urethral catheter of FIG. 9, and FIG. 12 is a drawing illustrating a cross-section of the urethra expansion part.

### BEST MODE

A drug-eluting expandable urethral balloon catheter of the present disclosure includes a tube part inserted into the urethra; a separation prevention part including a first balloon that inflates in the bladder to prevent separation of the tube part and maintain airtightness of the bladder; a drug injection part having a plurality of pores formed so that a drug is injected into a stricture site of the urethra; a urethra expansion part including a second balloon positioned and inflated at a front end of the drug injection part to prevent leakage of the drug; and a branched connection part branched from one side of the tube part to be connected thereto.

### MODE FOR INVENTION

Hereinafter, preferred embodiments of the present disclosure will be described in detail with reference to the attached drawings. Advantages and features of the present disclosure and methods for achieving them will become clear with reference to the embodiments described in detail below together with the attached drawings. However, the present disclosure is not limited to the embodiments disclosed below and may be implemented in various different forms. These embodiments are provided only to ensure that the present disclosure is complete and to fully inform those skilled in the art to which the present disclosure belongs of the scope of the present disclosure, and the present disclosure is defined only by the scope of the claims. Throughout the specification, identical reference numerals refer to identical components.

Although first, second, etc. are used to describe various elements, components and/or sections, these elements, components and/or sections are not limited by these terms. These terms are used only to distinguish one element, component or section other elements, from components or sections. Accordingly, it should be understood that the first element, first component or first section mentioned below may be the second element, second component or second section within the technical spirit of the present disclosure.

Terms used herein is for the purpose of describing embodiments only and is not intended to limit the present disclosure. In this specification, singular forms also include plural forms unless specifically stated otherwise in the text. Words “comprises” and/or “made of” as used in the specification indicate that component, a step, an operation and/or element which are mentioned do not exclude the presence or addition of one or more other components, steps, operations and/or elements.

Unless otherwise defined, all terms (including technical and scientific terms) used herein may be used as meanings commonly understood by those skilled in the art to which the present disclosure belongs. In addition, terms defined in commonly used dictionaries should not be interpreted ideally or excessively unless explicitly and specifically defined otherwise.

Hereinafter, the present disclosure will be described in more detail with reference to the accompanying drawings.

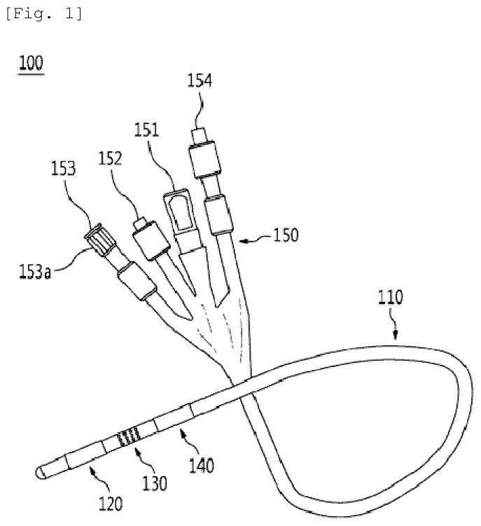

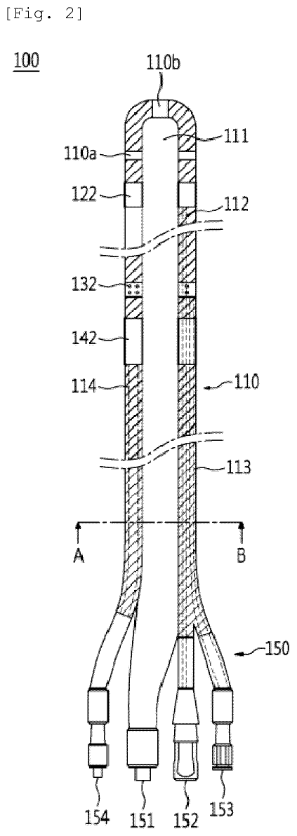

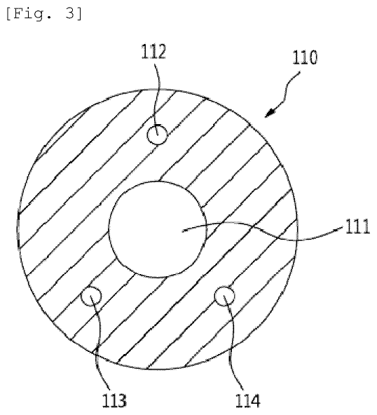

FIG. 1 is a drawing illustrating a drug-eluting expandable urethral balloon catheter according to an embodiment of the present disclosure. In addition, FIG. 2 is a drawing illustrating a longitudinal cross-section of the urethral catheter of FIG. 1. In addition, FIG. 3 is a drawing illustrating the A-B cross-section of a tube part of the urethral catheter of FIG. 2.

A drug-eluting expandable urethral balloon catheter 100 (hereinafter, referred to as “a urethral catheter”) according to an embodiment of the present disclosure includes a tube part 110 inserted into the urethra, a separation prevention part 120 including a first balloon 122 that inflates in the bladder to prevent separation of the tube part 110 and maintain airtightness of the bladder, a drug injection part 130 having a plurality of pores 132 formed so that a drug is injected into a stricture site of the urethra, a urethra expansion part 140 including a second balloon 142 positioned and inflated at a front end of the drug injection part 130 to prevent leakage of the drug, and a branched connection part 150 branched from one side of the tube part 110 to be connected thereto.-1

[symbol]

Referring to FIGS. 1 to 3, the tube part 110 corresponds to the main body of the urethral catheter 100 and is inserted into the urethra. More specifically, the tube part 110 is inserted into the urethra from the opposite side of the branched connection part 150, and a portion thereof is located outside the urethra.

The separation prevention part 120, the drug injection part 130, the urethra expansion part 140 may be formed in areas of the tube part 110, and the branched connection part 150 may be formed in another area of the tube part 110. The branched connection part 150 is positioned outside the urethra, the drug injection part 130 and the urethra expansion part 140 are inserted into and positioned inside the urethra, and the separation prevention part 120 is positioned inside the bladder. That is, the tube part 110 may be inserted through the urethra into the bladder.

The tube part 110 has excellent biocompatibility and may be formed of a flexible material. For example, the tube part 110 may be made of silicone, latex, etc. Additionally, the tube part 110 may additionally include an antibiotic to prevent infection. For example, the tube part 110 may additionally include a ZincO material.

A first inlet hole 110a and a second inlet hole 110b are formed on a distal end of the tube part 110 that is inserted into the bladder. The first inlet hole 110a and the second inlet hole 110b allow the inside of the bladder and the inside of the tube part 110 to communicate with each other so that urine may be discharged through the tube part 110.

More specifically, the first inlet hole 110a may be formed in a diametrical direction on a side of the distal end of the tube part 110, and the shape and number of the first inlet hole 110a are not particularly limited. For example, the first inlet hole 110a may be oval or circular, and may include a plurality of first inlet holes formed along the side of the tube part 110.

In addition, the second inlet hole 110b may be formed at a distal tip of the tube part 110. The shape of the distal tip of the tube part 110 is not particularly limited, but may be formed into a streamlined shape to prevent damage to tissues such as the urethra or bladder due to the insertion of the tube part 110. Since the second inlet hole 110b is formed in the central portion of the distal tip of the tube part 110, a guide wire, etc. may be inserted into the inside of the tube part 110 for use.

The tube part 110 may include a urinary tube 111, a first fluid passage tube 112, a drug injection tube 113, and a second fluid passage tube 114.

Here, the urinary tube 111 may be located between a first adapter 151 of the branched connection part 150 and the second inlet hole 110b to be connected thereto. A discharge hole for discharging urine may be formed on one side of the first adapter 151 of the branched connection part 150, and the first adapter 151 may be connected to a urine bag (not shown) for storing urine. The urine bag may be fastened and connected to one end of the first adapter 151 of the branched connection part 150. Accordingly, urine accumulated in the bladder of the patient may be discharged out of the body through the urinary tube 111.

Additionally, the first fluid passage tube 112 may communicate with the first balloon 122. More specifically, the first fluid passage tube 112 may be located between a second adapter 152 of the branched connection part 150 and the first balloon 122 to be connected thereto. Liquid or air is introduced into the first balloon 122 through the first fluid passage tube 112 to inflate the first balloon 122. In the bladder, the first balloon 122 may be inflated by the inflow of fluid through the first fluid passage tube 112, so that the first balloon 122 may be maintained within the bladder even with the movement of the urethral catheter 100. The material of the first balloon 122 is not particularly limited, but may be an elastic body that has excellent biocompatibility and can be easily inflated by injecting fluid.

In addition, the drug injection tube 113 may communicate with the plurality of pores 132. More specifically, the drug injection tube 113 may be located between a third adapter 153 of the branched connection part 150 and the pores 132 to be connected thereto. Since the drug injection part 130 is provided on the stricture site of the urethra so that drugs may be administered from the outside, the urethra expansion part 140 may expand the stricture site to prevent stricture, and at the same time, administer therapeutic drugs to the stricture site so that rapid treatment may be performed.

In this case, the drugs may be periodically sent through the drug injection tube 113 to the pores 132 and the drugs may be diffused from the pores 132 to the stricture site, so that a patient may perform drug therapy on his/her own.

In addition, the second fluid passage tube 114 may communicate with the second balloon 142. More specifically, the second fluid passage tube 114 may be located between a fourth adapter 154 of the branched connection part 150 and the second balloon 142 to be connected thereto. Liquid or air is introduced into the second balloon 142 through the second fluid passage tube 114 to inflate the second balloon 142. The material of the second balloon 142 is not particularly limited, but may be an elastic body that has excellent biocompatibility and can be easily inflated by injecting fluid.

In this case, fluid may be periodically sent to the second balloon 142 through the second fluid passage tube 114, and the second balloon 142 is inflated at the stricture site of the urethra to mechanically and physically suppress restenosis. Therefore, it is possible for patients to safely perform urethral dilation on their own periodically during the period in which the urethral catheter 100 is in place.

Referring back to FIGS. 2 and 3, the tube part 110 has a hollow interior constituting the urinary tube 111, and the first fluid passage tube 112, the drug injection tube 113, and the second fluid passage tube 114 may each extend within the cross-section of the tube part 110.

Referring back to FIG. 2, the urinary tube 111, the first fluid passage tube 112, the drug injection tube 113, and the second fluid passage tube 114 are connected to adapters 151, 152, 153, and 154 of the branched connection part 150, respectively.

That is, the urethral catheter 100 does not include the urinary tube 111 as a separate tube, and the urinary tube 111 itself constitutes an external tube.

In addition, although not shown in the drawing, at least one of the first fluid passage tube 112, the drug injection tube 113, and the second fluid passage tube 114 may extend in the hollow portion of the urinary tube 212 rather than within the cross-section of the urinary tube 111.

Alternatively, although not shown in the drawing, at least one of the first fluid passage tube 112, the drug injection tube 113, and the second fluid passage tube 114 may extend outside the urinary tube 212.

Referring back to FIG. 3, it is preferable that each of the first fluid passage tube 112, the drug injection tube 113, and the second fluid passage tube 114 has a cross-sectional diameter smaller than the cross-sectional diameter of the urinary tube 111. The first balloon 122 is inflated in the bladder to prevent the separation of the separation prevention part 120 by restricting the movement the separation prevention part 120 and to maintain the airtightness of the inside of the bladder. The first balloon 122 is connected to the first fluid passage tube 112 to perform the separation prevention function.

In the drug injection part 130, drug passing through the plurality of pores 132 spreads to the stricture site, thereby preventing urethral stricture. The plurality of pores 132 are connected to the drug injection tube 113 and perform a drug injection function. Drug may be injected periodically through the drug injection tube 113 and the plurality of pores 132.

The second balloon 142 of the urethra expansion part 140 is inflated at the stricture site of the urethra to stimulate a surface tissue and expand the urethra, thereby preventing urethral stricture. The second balloon 142 is connected to the second fluid passage tube 114 to expand the space of the stricture site of the urethra. In this case, a physical method for dilating the urethra and drug therapy for injecting a drug may be performed simultaneously.

The branched connection part 150 is branched from one side of the tube part to be connected thereto, and liquid, air, drugs, etc. may be introduced or discharged through the first fluid passage tube 112, the drug injection tube 113, and the second fluid passage tube 114 of the tube part 110. To perform this function, the branched connection part 150 may include the plurality of adapters.

For example, the branched connection part 150 may include the first adapter 151 which performs urinary catheterization from the bladder 15, the second adapter 152 which introduces fluid, such as air or saline solution, into the first balloon 122, the third adapter 153 which injects a drug into the stricture site of the urethra through the plurality of pores 132, and the fourth adapter 154 which introduces or discharges fluid, such as air or saline solution, into or out of the second balloon 142.

That is, the first adapter 151 may be connected to the urinary tube 111 to quickly discharge urine accumulated in the bladder. In addition, the second adapter 152 may be connected to the first fluid passage tube 112 to inject fluid, such air or saline solution, into the first balloon 122. In addition, the third adapter 153 may be connected to the drug injection tube 113 to inject drugs into the plurality of pores. In addition, the fourth adapter 154 may be connected to the second fluid passage tube 114 to inject fluid, such as air, into the first balloon 142.

Particularly, the branched connection part 150 may simultaneously perform balloon dilation of the urethra and drug injection into the urethra by inflating the second balloon 142 by introducing fluid thereto through the fourth adapter 154 when injecting a drug into the stricture site of the urethra through the third adapter 153.

Here, a syringe, etc. may be connected to each end of the second adapter 152, the third adapter 153, and the fourth adapter 154 so that a patient may periodically dilate the urethra and inject a drug thereto on his/her own. Particularly, to prevent the leak of a drug to the outside and the inflow of external air after the patient has administered the drug on his/her own, the third adapter 153 may be provided with a protective cap 153a.

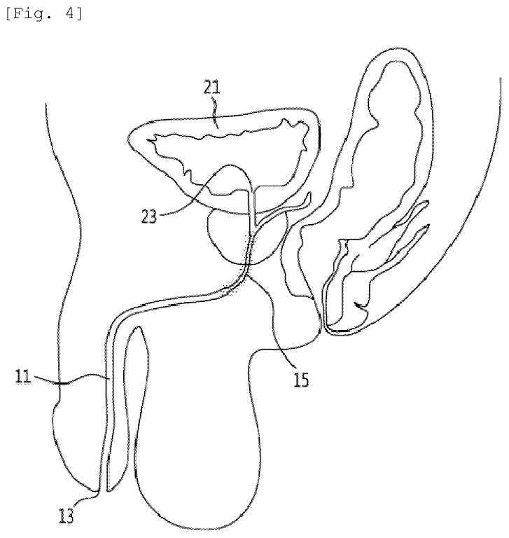

FIG. 4 is a drawing illustrating the body into which the urethral catheter is inserted, and FIG. 5 is a drawing illustrating a state in which the urethral catheter is inserted.

Referring to FIGS. 4 and 5, the urethral catheter 100 is inserted through the urethra 11 and reaches the bladder 21. When the urethral catheter 100 is inserted, an entrance 13 of the urethra 11 is blocked, and when the first balloon 122 is inflated, an entrance 23 of the bladder 21 also is blocked.

After the urethral catheter 100 is placed, it is possible to perform a urethral dilation procedure by inflating the second balloon 142 at the stricture site 15 of the urethra 11, and it is possible to perform drug therapy by releasing a drug into the stricture site 15 through the pores 132.

Accordingly, a patient may periodically perform ballooning of the second balloon 142 on his or her own and inject a drug into the stricture site 15 from outside the urethra, thereby increasing patient convenience and greatly increasing the effect of preventing restenosis.

Although not shown in the drawing, the pores 132 may be formed not only in the tube part 110 corresponding to the main body, but also in the second balloon 142.

Alternatively, the pores 132 may be formed only in the second balloon 142.

In addition, although not shown in the drawing, a plurality of protrusions may be formed on the surface of the second balloon 142. By forming the plurality of protrusions on the surface of the second balloon 142, microscopic stimulation may be provided to the surface tissue of the urethra when the second balloon 142 is inflated. These protrusions form tiny gaps in the surface tissue of the urethra, allowing a drug to remain well between the gaps. Through this, the rate of drug penetration into the urethral tissue may be increased.

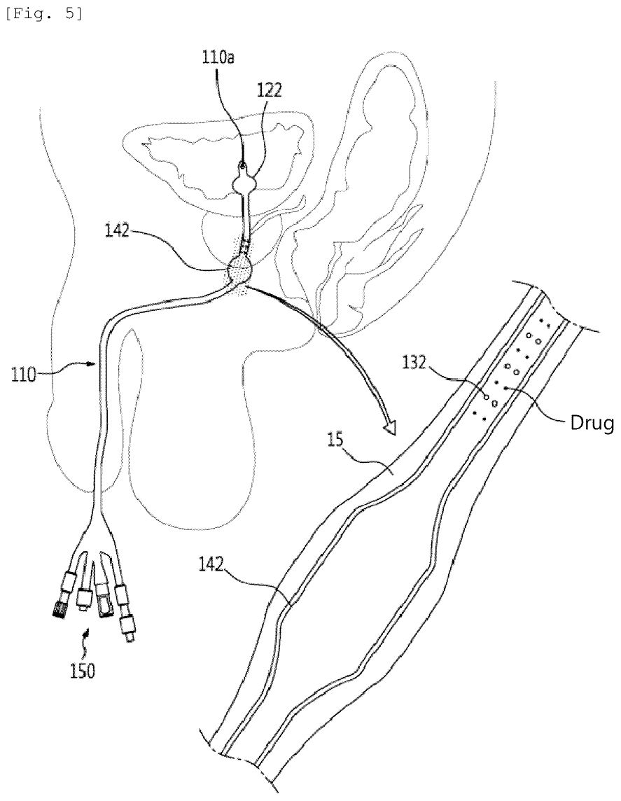

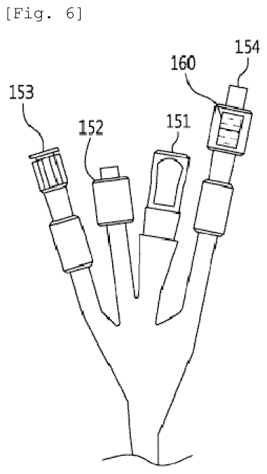

FIG. 6 is a drawing illustrating a sensing part installed in the urethral catheter of FIG. 1. In addition, FIG. 7 is a drawing illustrating the detailed configuration of the sensing part of FIG. 6.

Referring to FIGS. 6 and 7, the urethral catheter 100 may further include a sensing part 160. The sensing part 160 senses the pressure of fluid flowing into or out of the second fluid passage tube 154 and provides data on the sensed pressure.

Specifically, the sensing part 160 may include a sensing module 162, a control module 164, and a display module 166, etc.

The sensing module 162 is installed in a fluid moving passage of the second fluid passage tube 154 and senses the pressure of fluid.

The control module 164 processes data from the sensing module 162 and controls the sensing module 162, and the display module 166, etc.

The display module 166 displays data on pressure measured by the sensing module 162 to guide a user of the urethral catheter 100. It is preferable that the display module 166 is installed in the fourth adapter 154, which is connected to one end of the second fluid passage tube 154, so that a user may quickly and intuitively check the pressure. The display module 166 may be applied to display in various forms, such as a digital number or a pressure gauge.

In this case, the control module 164 may process the data on pressure measured by the sensing module 162 and control the display of the data on the display module 166.

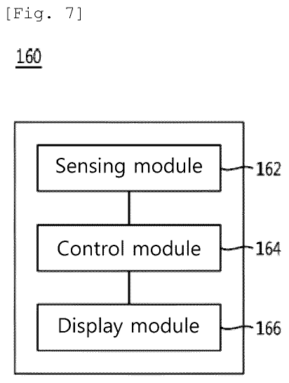

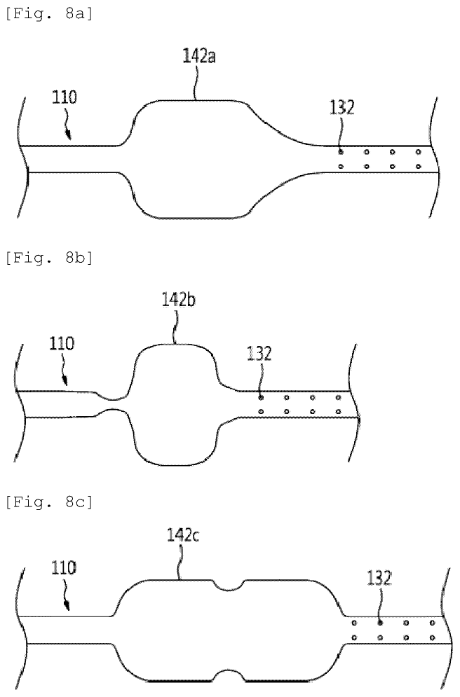

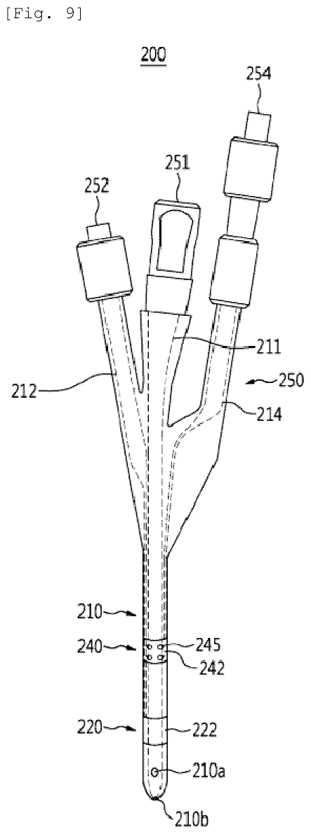

FIGS. 8a to 8c are drawings illustrating several embodiments of a second balloon of the urethral catheter of FIG. 1.

Referring to FIGS. 8a to 8c, various embodiments of the second balloon 142 are illustrated, wherein three embodiments of second balloons 142a, 142b, and 142c may reliably prevent a drug released from the pores 132 from being discharged out of the urethra.

As illustrated in FIG. 8a, when the second balloon 142a of the first embodiment is inflated, a portion of the second balloon 142a adjacent to the tube part 110 may be formed to be inflated steeper than a portion of the second balloon 142a adjacent to the drug injection part 130. More specifically, after the diameter of the second balloon 142a gradually increases from the portion adjacent to the drug injection part 130, the diameter of the second balloon 142a may be constant, and then the diameter of the second balloon 142a may decrease again. In this case, with reference to the distance of the drug injection part 130, the slope of the portion in which the diameter of the second balloon 142a gradually decreases may be formed to be steeper than that of the portion in which the diameter of the second balloon 142a gradually increases.

Accordingly, since the diameter of the second balloon 142a gradually increases from the portion adjacent to the drug injection part 130, a drug released from the pores 132 may be prevented from flowing toward the entrance of the urethra. In addition, the diameter of the second balloon 142a at the portion adjacent to the tube part 110 is greater than the diameter thereof at the portion adjacent to the drug injection part 130, and accordingly, when the urethral catheter 100 is removed from the urethra, the portion in which the diameter of the second balloon 142a is greater may be pressed with greater pressure, thereby allowing the removal to be more comfortable.

As illustrated in FIG. 8b, when the second balloon 142b of the second embodiment is inflated, the thickness of a portion of the tube part 110 connected to the second balloon 142b may be small.

Accordingly, the drug released from the pores 132 may be prevented from flowing toward the entrance of the urethra, and the thickness of the connecting portion of the second balloon 142b and the tube part 110 may be small, thereby further r increasing a force applied to the second balloon 142b. Through this, by reducing the diameter of the second balloon 142b by the force pulling the urethral catheter 100, the urethral catheter 100 may be more easily removed from the urethra.

As illustrated in FIG. 8c, when the second balloon 142c of the third embodiment is inflated, a groove may be formed in a portion of the second balloon 142c so that the second balloon 142c may be divided into two parts.

Accordingly, a drug released from the pores 132 may be reliably prevented from flowing toward the entrance of the urethra by the tapered structure formed in the second balloon 142c of the third embodiment. In addition, the flow of the drug may be prevented once more by the middle tapered structure, and thus when the second balloon 142c of the third embodiment is inflated, a maximum diameter thereof may be smaller than that of each of 142a and 142b of the first and second embodiments.

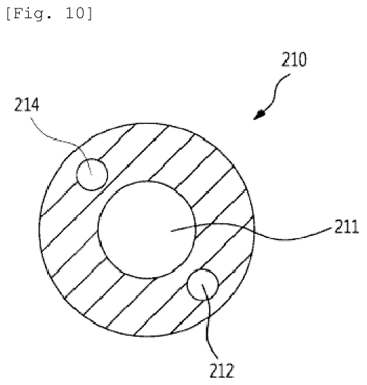

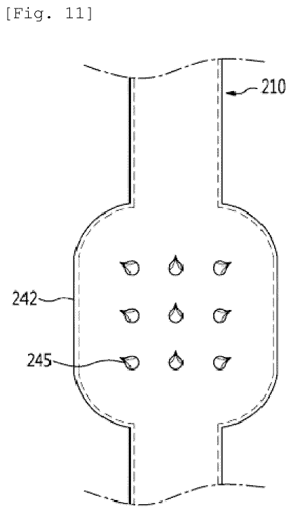

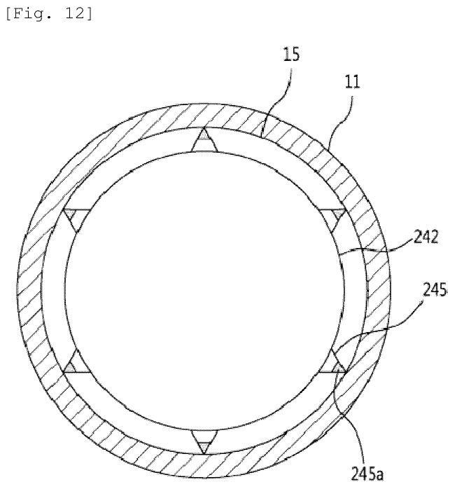

FIG. 9 is a drawing illustrating drug-eluting expandable urethral balloon catheter according to another embodiment of the present disclosure. In addition, FIG. 10 is a drawing illustrating a cross-section of the tube part of the urethral catheter of FIG. 9. In addition, FIG. 11 is a drawing illustrating microneedles formed in a urethra expansion part of the urethral catheter of FIG. 9, and FIG. 12 is a drawing illustrating a cross-section of the urethra expansion part.

Referring to FIG. 9, a drug-eluting expandable urethral balloon catheter 200 (hereinafter, referred to as “a urethral catheter”) according to another embodiment of the present disclosure includes a tube part 210 inserted into the urethra, a separation prevention part 220 including a third balloon 222 that inflates in the bladder to prevent separation of the tube part 210 and maintain airtightness of the bladder, a urethra expansion part 240 including a fourth balloon 242 positioned and inflated at a stricture site of the urethra to prevent leakage of a drug, and a branched connection part 250 branched from one side of the tube part 210 to be connected thereto. Here, the urethra expansion part 240 has a plurality of microneedles 245 formed on the surface of the fourth balloon 242.

Referring to FIG. 10, in a urethral catheter 200 according to another embodiment of the present disclosure, the tube part 210 includes a urinary tube 211, a third fluid passage tube 212 communicating with the third balloon 222, and a fourth fluid passage tube 214 communicating with the fourth balloon 242.

In this case, a hollow portion of the tube part 210 constitutes the urinary tube 211, and each of the third fluid passage tube 212 and the fourth fluid passage tube 214 extends within the cross-section of the tube part 210. The tube part 210 includes inlet holes 210a and 210b on a distal end thereof.

In addition, the urinary tube 211, the third fluid passage tube 212, and the fourth fluid passage tube 214 are connected to respective adapters 251, 252, and 254 located in the branched connection part 250. This has been explained above, and thus a detailed explanation thereof will be omitted.

Referring to FIG. 11, the urethra expansion part 240 may have a plurality of microneedles 245 formed on the surface of the fourth balloon 242 thereof. The microneedles 245 are formed on the surface of the fourth balloon 242, and thus micro-stimulate the surface tissue of the urethra when the fourth balloon 242 is inflated, thereby facilitating drug penetration and allowing a drug to remain well between tiny gaps. That is, the microneedles 245 may function as tiny punctures on the entire surface of the urethra, thereby increasing the rate of the drug penetration into the tissue. Referring to FIG. 12, the microneedles 245 of the urethra expansion part 240 may include an antifibrotic drug 245a. That is, a tip made of the antifibrotic drug 245a may be formed on the end of the microneedles 245. The antifibrotic drug 245a may be a soluble drug such as hyaluronic acid. As the fourth balloon 242 is inflated, the microneedles 245 stimulate the surface tissue of the stricture site 15 of the urethra 11, and the antifibrotic drug 245a is dissolved, allowing the drug to penetrate well into the tissue.

Although the embodiments of the present disclosure has been described with reference to the attached drawings, those skilled in the art to which the present disclosure pertains will understand that the present disclosure may be implemented in other specific forms without changing the technical idea or essential features thereof. Therefore, it should be understood that the embodiments described above are illustrative in all respects and not limiting.

### INDUSTRIAL APPLICABILITY

Applicability to the field of treating urological diseases may be provided.

1. A drug-eluting expandable urethral balloon catheter comprising:
a tube part inserted into the urethra;
a separation prevention part comprising a first balloon that inflates in the bladder to prevent separation of the tube part and maintain airtightness of the bladder;
a drug injection part having a plurality of pores formed so that a drug is injected into a stricture site of the urethra;
a urethra expansion part comprising a second balloon positioned and inflated at a front end of the drug injection part to prevent leakage of the drug; and
a branched connection part branched from one side of the tube part to be connected thereto.

2. The catheter of claim 1, wherein the tube part comprises:
a urinary tube;
a first fluid passage tube communicating with the first balloon;
a drug injection tube communicating with the plurality of pores; and
a second fluid passage tube communicating with the second balloon.

3. The catheter of claim 2, wherein the tube part has a hollow portion constituting the urinary tube, and
each of the first fluid passage tube, the drug injection tube, and the second fluid passage tube extends within a cross-section of the tube part.

4. The catheter of claim 1, wherein the branched connection part comprises:
a first adapter connected to a urinary tube of the tube part to perform urinary catheterization from the bladder;
a second adapter connected to introduce or discharge fluid, such as air or saline solution, into or out of the first balloon;
a third adapter connected to inject a drug into the stricture site of the urethra through the plurality of pores; and
a fourth adapter connected to introduce or discharge fluid, such as air or saline solution, into or out of the second balloon.

5. The catheter of claim 4, wherein the branched connection part inflates the second balloon by introducing fluid thereto through the fourth adapter when injecting a drug into the stricture site of the urethra through the third adapter.

6. The catheter of claim 1, wherein when the second balloon of the urethra expansion part is inflated, a portion of the second balloon adjacent to the tube part is formed to be inflated steeper than a portion of the second balloon adjacent to the drug injection part.

7. The catheter of claim 1, wherein when the second balloon is inflated, a thickness of a portion of the tube part connected to the second balloon is small.

8. The catheter of claim 1, wherein when the second balloon of the urethra expansion part is inflated, a groove is formed in a portion of the second balloon so that the second balloon is divided into two parts.

9. The catheter of claim 2, further comprising:
a sensing part which senses pressure of fluid flowing into or out of the first fluid passage tube and the second fluid passage tube.

10. A drug-eluting expandable urethral balloon catheter comprising:
a tube part inserted into the urethra;
a separation prevention part comprising a third balloon that inflates in the bladder to prevent separation of the tube part and maintain airtightness of the bladder;
a urethra expansion part comprising a fourth balloon positioned and inflated at a stricture site of the urethra to prevent leakage of a drug; and
a branched connection part branched from one side of the tube part to be connected thereto,
wherein the urethra expansion part has a plurality of microneedles formed on a surface of the fourth balloon.

11. The catheter of claim 10, wherein the microneedles of the urethra expansion part comprises an antifibrotic drug.
